(19) United States
(12) Patent Application Publication
(10) Pub. No.: US 2023/0210627 A1
(43) Pub. Date: Jul. 6, 2023

(54) THREE-DIMENSIONAL INSTRUMENT POSE ESTIMATION

(71) Applicant: Auris Health, Inc., Redwood City, CA (US)

(72) Inventors: Elif AYVALI, Redwood City, CA (US); Bulat Ibragimov, Copenhagen (DK); Jialu Li, Sunnyvale, CA (US)

(73) Assignee: Auris Health, Inc., Redwood City, CA (US)

(21) Appl. No.: 18/067,688

(22) Filed: Dec. 16, 2022

Related U.S. Application Data
(60) Provisional application No. 63/295,515, filed on Dec. 31, 2021.

(51) Int. Cl. A61B 90/00; A61B 34/20

(52) U.S. Cl. CPC A61B 90/36; A61B 34/20; A61B 2034/2059; A61B 2034/2065; A61B 2090/367

(57) ABSTRACT

The present disclosure relates to systems, devices, and methods for augmenting a two-dimensional image with three-dimensional pose information of instruments shown in the two-dimensional image.

### RELATED APPLICATION(S)

This application claims priority to U.S. Provisional Application No. 63/295,515, filed Dec. 31, 2021, entitled THREE-DIMENSIONAL INSTRUMENT POSE ESTIMATION, the disclosure of which is hereby incorporated by reference in its entirety.

### BACKGROUND

Various medical procedures involve the use of one or more devices configured to penetrate the human anatomy to reach a treatment site. Certain operational processes can involve localizing a medical instrument within the patient and visualizing an area of interest within the patient. To do so, many medical instruments may include sensors to track the location of the instrument and may include vision capabilities, such as embedded cameras or the compatible use with vision probes.

### BRIEF DESCRIPTION OF THE DRAWINGS

Various embodiments are depicted in the accompanying drawings for illustrative purposes and should in no way be interpreted as limiting the scope of the disclosure. In addition, various features of different disclosed embodiments can be combined to form additional embodiments, which are part of this disclosure. Throughout the drawings, reference numbers may be reused to indicate correspondence between reference elements.

FIG. 1 is a block diagram that illustrates an example medical system for performing various medical procedures in accordance with aspects of the present disclosure.

FIG. 2 is a block diagram illustrating the modules implemented by the augmentation module and the data stored in the data store of FIG. 1, according to an example embodiment.

FIG. 3 is a flow-chart illustrating a method to visualize a three-dimensional pose of a first instrument with respect to a two-dimensional image, according to an example embodiment.

FIG. 4 is a block diagram illustrating a segmentation of two-dimensional image data, according to an example embodiment.

FIG. 5 is a diagram illustrating a cart system, according to an example embodiment.

FIG. 6 is a diagram illustrating an example of an augmented representation of two-dimensional image data, consistent with example embodiments contemplated by this disclosure.

FIG. 7 is a diagram illustrating an example of an augmented representation of two-dimensional image data, consistent with example embodiments contemplated by this disclosure.

FIG. 8 is a diagram illustrating an example of an augmented representation of two-dimensional image data, consistent with example embodiments contemplated by this disclosure.

FIG. 9 is a diagram illustrating an example of an augmented representation of two-dimensional image data, consistent with example embodiments contemplated by this disclosure.

### DETAILED DESCRIPTION

The headings provided herein are for convenience only and do not necessarily affect the scope or meaning of disclosure. Although certain exemplary embodiments are disclosed below, the subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

### Overview

The present disclosure relates to systems, devices, and methods for augmenting a two-dimensional image with three-dimensional pose information of an instrument or instruments shown in the two-dimensional image. Such information of a three-dimensional pose may be provided relative to a two-dimensional image.

Providing information relating to three-dimensional pose of an instrument relative to a two-dimensional image can have practical applications. As explained later in this disclosure, some embodiments of a medical system may allow for an operator to perform a percutaneous procedure where one instrument (e.g., a needle) attempts to rendezvous with another (e.g., an instrumented scope). In attempting to rendezvous one instrument with another, an operator of the medical system may acquire a fluoroscopic image to verify that the instruments are advancing in an expected manner. However, a two-dimensional image like a fluoroscopic image may be insufficient in confirming whether the instruments are advancing in a suitable manner because the operator of the medical system would benefit from three-dimensional context that is lacking in a fluoroscopic image.

By way of further example and not limitation, a medical system may also allow an operator to navigate an instrument that lacks vision capability, either because the instrument lacks vision capabilities itself or because the instrument is outside a viewing range of a camera operated by the system. This may be referred to as blind driving. It's important in these medical systems for the operator to drive safely within sensitive anatomy and avoid unsafe contact with anatomy or other instruments that may be in the operating space of the procedure. In these blind driving situations, a fluoro image may be captured to get a sense of the positioning of the scope relative to the anatomy or other instruments. But, similar to the rendezvous procedure discussed above, the fluoro image may lack three-dimensional context information regarding the pose of the instruments captured in the fluoro image.

Embodiments discussed herein may use system data, such as sensor data and robotic data to generate an augmented representation of the two-dimensional image such that the augmented representation includes a three-dimensional representation of the instruments shown relative to the two-dimensional image. Doing so may provide an operator with three-dimensional context in which to navigate or otherwise control an instrument using a two-dimensional image in which the operator is comfortable in reviewing.

### Three-Dimensional Pose Estimation System

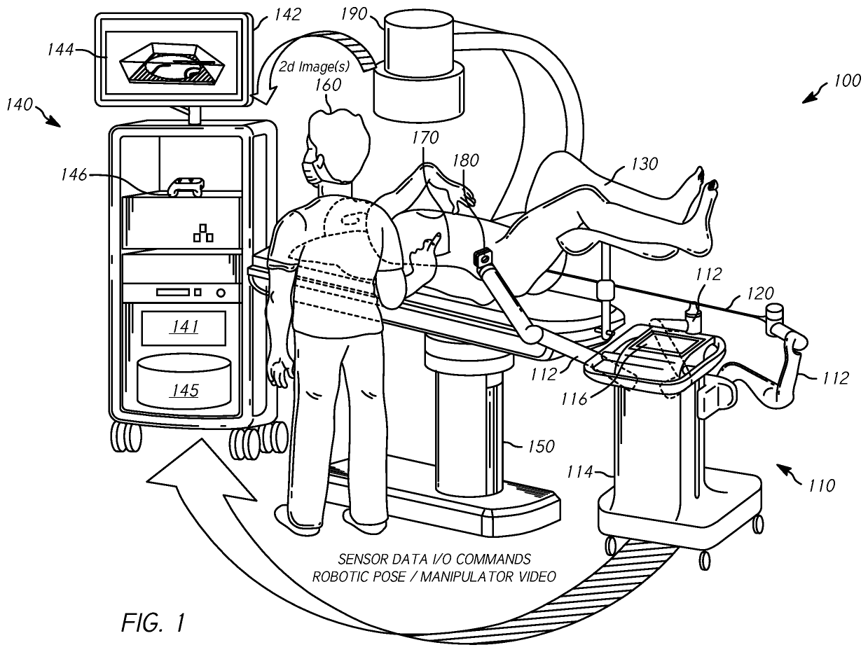

FIG. 1 is a block diagram that illustrates an example medical system 100 for performing various medical procedures in accordance with aspects of the present disclosure. The medical system 100 includes a robotic system 110 configured to engage with and/or control a medical instrument 120 to perform a procedure on a patient 130. The medical system 100 also includes a control system 140 configured to interface with the robotic system 110, provide information regarding the procedure, and/or perform a variety of other operations. For example, the control system 140 can include a display(s) 142 to present certain information to assist the physician 160. The display(s) 142 may be a monitor, screen, television, virtual reality hardware, augmented reality hardware, three-dimensional imaging devices (e.g., hologram devices) and the like, or combinations thereof. The medical system 100 can include a table 150 configured to hold the patient 130. The system 100 can further include an electromagnetic (EM) field generator 180, which can be held by one or more robotic arms 112 of the robotic system 110 or can be a stand-alone device. In examples, the medical system 100 can also include an imaging device 190 which can be integrated into a C-arm and/or configured to provide imaging during a procedure, such as for a fluoroscopy-type procedure.

In some implementations, the medical system 100 can be used to perform a percutaneous procedure. For example, if the patient 130 has a kidney stone that is too large to be removed through a urinary tract, the physician 160 can perform a procedure to remove the kidney stone through a percutaneous access point on the patient 130. To illustrate, the physician 160 can interact with the control system 140 to control the robotic system 110 to advance and navigate the medical instrument 120 (e.g., a scope) from the urethra, through the bladder, up the ureter, and into the kidney where the stone is located. The control system 140 can provide information via the display(s) 142 regarding the medical instrument 120 to assist the physician 160 in navigating the medical instrument 120, such as real-time images captured therewith.

Once at the site of the kidney stone (e.g., within a calyx of the kidney), the medical instrument 120 can be used to designate/tag a target location for the medical instrument 170 (e.g., a needle) to access the kidney percutaneously (e.g., a desired point to access the kidney). To minimize damage to the kidney and/or the surrounding anatomy, the physician 160 can designate a particular papilla as the target location for entering into the kidney with the medical instrument 170. However, other target locations can be designated or determined. To assist the physician in driving the medical instrument 170 into the patient 130 through the particular papilla, the control system 140 can provide an augmented visualization interface 144, which can include a rendering of an augmented visualization of a two-dimensional image captured by the system 100, such as a fluoroscopic image. The augmented visualization can include a three-dimensional representation of the instrument 170 in conjunction with a planar representation of two-dimensional image data acquired by the imaging device 190. As is explained in greater detail, the augmented visualization interface 144 may provide information to the operator that is helpful in driving the medical instrument 170 to the target location.

Once the instrument 170 has reached the target, the physician 160 can use the medical instrument 170 and/or another medical instrument to extract the kidney stone from the patient 130. One such instrument may be a percutaneous catheter. The percutaneous catheter may be an instrument with steering capabilities, much like the instrument 120, but may, in some embodiments, lack a dedicated camera or location sensor. Some embodiments may use the augmented visualization interface 144 to render augmented images that are helpful in driving the percutaneous catheter within the anatomy.

Although the above percutaneous procedure and/or other procedures are discussed in the context of using the medical instrument 120, in some implementations a percutaneous procedure can be performed without the assistance of the medical instrument 120. Further, the medical system 100 can be used to perform a variety of other procedures.

Moreover, although many embodiments describe the physician 160 using the medical instrument 170, the medical instrument 170 can alternatively be used by a component of the medical system 100. For example, the medical instrument 170 can be held/manipulated by the robotic system 110 (e.g., the one or more robotic arms 112) and the techniques discussed herein can be implemented to control the robotic system 110 to insert the medical instrument 170 with the appropriate pose (or aspect of a pose, such as orientation or position) to reach a target location.

In the example of FIG. 1, the medical instrument 120 is implemented as a scope and the medical instrument 170 is implemented as a needle. Thus, for ease of discussion, the medical instrument 120 is referred to as “the scope 120” or “the lumen-based medical instrument 120,” and the medical instrument 170 is referred to as “the needle 170” or “the percutaneous medical instrument 170.” However, the medical instrument 120 and the medical instrument 170 can each be implemented as a suitable type of medical instrument including, for example, a scope (sometimes referred to as an “endoscope”), a needle, a catheter, a guidewire, a lithotripter, a basket retrieval device, forceps, a vacuum, a needle, a scalpel, an imaging probe, jaws, scissors, graspers, needle holder, micro dissector, staple applier, tacker, suction/irrigation tool, clip applier, and so on. In some embodiments, a medical instrument is a steerable device, while other embodiments a medical instrument is a non-steerable device. In some embodiments, a surgical tool refers to a device that is configured to puncture or to be inserted through the human anatomy, such as a needle, a scalpel, a guidewire, and so on. However, a surgical tool can refer to other types of medical instruments.

In some embodiments, a medical instrument, such as the scope 120 and/or the needle 170, includes a sensor that is configured to generate sensor data, which can be sent to another device. In examples, sensor data can indicate a location/orientation of the medical instrument and/or can be used to determine a location/orientation of the medical instrument. For instance, a sensor can include an electromagnetic (EM) sensor with a coil of conductive material. Here, an EM field generator, such as the EM field generator 180, can provide an EM field that is detected by the EM sensor on the medical instrument. The magnetic field can induce small currents in coils of the EM sensor, which can be analyzed to determine a distance and/or angle/orientation between the EM sensor and the EM field generator. Further, a medical instrument can include other types of sensors configured to generate sensor data, such as one or more of any of: a camera, a range sensor, a radar device, a shape sensing fiber, an accelerometer, a gyroscope, a satellite-based positioning sensor (e.g., a global positioning system (GPS)), a radio-frequency transceiver, and so on. In some embodiments, a sensor is positioned on a distal end of a medical instrument, while in other embodiments a sensor is positioned at another location on the medical instrument. In some embodiments, a sensor on a medical instrument can provide sensor data to the control system 140 and the control system 140 can perform one or more localization techniques to determine/track a position and/or an orientation of a medical instrument.

In some embodiments, the medical system 100 may record or otherwise track the runtime data that is generated during a medical procedure. This runtime data may be referred to as system data. For example, the medical system 100 may track or otherwise record the sensor readings (e.g., sensor data) from the instruments (e.g., the scope 120 and the needle 170) in data store 145A (e.g., a computer storage system, such as computer readable memory, database, filesystem, and the like). In addition to sensor data, the medical system 100 can store other types of system data in data store 145. For example, in the context of FIG. 1, the system data can further include time series data of the video images captured by the scope 120, status of the robotic system 110, commanded data from an I/O device(s) (e.g., I/O device(s) 146 discussed below), audio data (e.g., as may be captured by audio capturing devices embedded in the medical system 100, such as microphones on the medical instruments, robotic arms, or elsewhere in the medical system), external (relative to the patient) imaging device (such as RGB cameras, LIDAR imaging sensors, fluoroscope imaging sensors, etc.), and image data from the imaging device 190, and the like.

As shown in FIG. 1, the control system 140 includes an augmentation module 141 which may be control circuitry configured to operate on the system data and the two-dimensional image data stored in the case data store 145 to generate an augmented representation of the two-dimensional image data with three-dimensional pose data. As is discussed in greater detail below, the augmentation module 141 may employ machine learning techniques to segment two-dimensional image data according to the instruments present in the two-dimensional images. In some embodiments, once the two-dimensional image data has been segmented, the augmentation module 141 may generate three-dimensional representations of the segmented instruments using other system data, such as location sensor data and robotic data.

The term “scope” or “endoscope” are used herein according to their broad and ordinary meanings and can refer to any type of elongate medical instrument having image generating, viewing, and/or capturing functionality and configured to be introduced into any type of organ, cavity, lumen, chamber, and/or space of a body. For example, references herein to scopes or endoscopes can refer to a ureteroscope (e.g., for accessing the urinary tract), a laparoscope, a nephroscope (e.g., for accessing the kidneys), a bronchoscope (e.g., for accessing an airway, such as the bronchus), a colonoscope (e.g., for accessing the colon), an arthroscope (e.g., for accessing a joint), a cystoscope (e.g., for accessing the bladder), a borescope, and so on.

A scope can comprise a tubular and/or flexible medical instrument that is configured to be inserted into the anatomy of a patient to capture images of the anatomy. In some embodiments, a scope can accommodate wires and/or optical fibers to transfer signals to/from an optical assembly and a distal end of the scope, which can include an imaging device, such as an optical camera. The camera/imaging device can be used to capture images of an internal anatomical space, such as a target calyx/papilla of a kidney. A scope can further be configured to accommodate optical fibers to carry light from proximately-located light sources, such as light-emitting diodes, to the distal end of the scope. The distal end of the scope can include ports for light sources to illuminate an anatomical space when using the camera/imaging device. In some embodiments, the scope is configured to be controlled by a robotic system, such as the robotic system 110. The imaging device can comprise an optical fiber, fiber array, and/or lens. The optical components can move along with the tip of the scope such that movement of the tip of the scope results in changes to the images captured by the imaging device.

A scope can be articulable, such as with respect to at least a distal portion of the scope, so that the scope can be steered within the human anatomy. In some embodiments, a scope is configured to be articulated with, for example, five or six degrees of freedom, including X, Y, Z coordinate movement, as well as pitch, yaw, and roll. A position sensor(s) of the scope can likewise have similar degrees of freedom with respect to the position information they produce/provide. A scope can include telescoping parts, such as an inner leader portion and an outer sheath portion, which can be manipulated to telescopically extend the scope. A scope, in some instances, can comprise a rigid or flexible tube, and can be dimensioned to be passed within an outer sheath, catheter, introducer, or other lumen-type device, or can be used without such devices. In some embodiments, a scope includes a working channel for deploying medical instruments (e.g., lithotripters, basketing devices, forceps, etc.), irrigation, and/or aspiration to an operative region at a distal end of the scope.

The robotic system 110 can be configured to at least partly facilitate execution of a medical procedure. The robotic system 110 can be arranged in a variety of ways depending on the particular procedure. The robotic system 110 can include the one or more robotic arms 112 configured to engage with and/or control the scope 120 to perform a procedure. As shown, each robotic arm 112 can include multiple arm segments coupled to joints, which can provide multiple degrees of movement. In the example of FIG. 1, the robotic system 110 is positioned proximate to the patient's 130 legs and the robotic arms 112 are actuated to engage with and position the scope 120 for access into an access point, such as the urethra of the patient 130. When the robotic system 110 is properly positioned, the scope 120 can be inserted into the patient 130 robotically using the robotic arms 112, manually by the physician 160, or a combination thereof. The robotic arms 112 can also be connected to the EM field generator 180, which can be positioned near a treatment site, such as within proximity to the kidneys of the patient 130.

The robotic system 110 can also include a support structure 114 coupled to the one or more robotic arms 112. The support structure 114 can include control electronics/circuitry, one or more power sources, one or more pneumatics, one or more optical sources, one or more actuators (e.g., motors to move the one or more robotic arms 112), memory/data storage, and/or one or more communication interfaces. In some embodiments, the support structure 114 includes an input/output (I/O) device(s) 116 configured to receive input, such as user input to control the robotic system 110, and/or provide output, such as a graphical user interface (GUI), information regarding the robotic system 110, information regarding a procedure, and so on. The I/O device(s) 116 can include a display, a touchscreen, a touchpad, a projector, a mouse, a keyboard, a microphone, a speaker, etc. In some embodiments, the robotic system 110 is movable (e.g., the support structure 114 includes wheels) so that the robotic system 110 can be positioned in a location that is appropriate or desired for a procedure. In other embodiments, the robotic system 110 is a stationary system. Further, in some embodiments, the robotic system 112 is integrated into the table 150.

The robotic system 110 can be coupled to any component of the medical system 100, such as the control system 140, the table 150, the EM field generator 180, the scope 120, and/or the needle 170. In some embodiments, the robotic system is communicatively coupled to the control system 140. In one example, the robotic system 110 can be configured to receive a control signal from the control system 140 to perform an operation, such as to position a robotic arm 112 in a particular manner, manipulate the scope 120, and so on. In response, the robotic system 110 can control a component of the robotic system 110 to perform the operation. In another example, the robotic system 110 is configured to receive an image from the scope 120 depicting internal anatomy of the patient 130 and/or send the image to the control system 140, which can then be displayed on the display(s) 142. Furthermore, in some embodiments, the robotic system 110 is coupled to a component of the medical system 100, such as the control system 140, in such a manner as to allow for fluids, optics, power, or the like to be received therefrom. Example details of the robotic system 110 are discussed in further detail below in reference to FIG. 12.

The control system 140 can be configured to provide various functionality to assist in performing a medical procedure. In some embodiments, the control system 140 can be coupled to the robotic system 110 and operate in cooperation with the robotic system 110 to perform a medical procedure on the patient 130. For example, the control system 140 can communicate with the robotic system 110 via a wireless or wired connection (e.g., to control the robotic system 110 and/or the scope 120, receive an image(s) captured by the scope 120, etc.), provide fluids to the robotic system 110 via one or more fluid channels, provide power to the robotic system 110 via one or more electrical connections, provide optics to the robotic system 110 via one or more optical fibers or other components, and so on. Further, in some embodiments, the control system 140 can communicate with the needle 170 and/or the scope 170 to receive sensor data from the needle 170 and/or the endoscope 120 (via the robotic system 110 and/or directly from the needle 170 and/or the endoscope 120). Moreover, in some embodiments, the control system 140 can communicate with the table 150 to position the table 150 in a particular orientation or otherwise control the table 150. Further, in some embodiments, the control system 140 can communicate with the EM field generator 180 to control generation of an EM field around the patient 130.

The control system 140 includes various I/O devices configured to assist the physician 160 or others in performing a medical procedure. In this example, the control system 140 includes an I/O device(s) 146 that is employed by the physician 160 or other user to control the scope 120, such as to navigate the scope 120 within the patient 130. For example, the physician 160 can provide input via the I/O device(s) 146 and, in response, the control system 140 can send control signals to the robotic system 110 to manipulate the scope 120. Although the I/O device(s) 146 is illustrated as a controller in the example of FIG. 1, the I/O device(s) 146 can be implemented as a variety of types of I/O devices, such as a touchscreen, a touch pad, a mouse, a keyboard, a surgeon or physician console, virtual reality hardware, augmented hardware, microphone, speakers, haptic devices, and the like.

As also shown in FIG. 1, the control system 140 can include the display(s) 142 to provide various information regarding a procedure. As noted above, the display(s) 142 can present the augmented visualization interface 144 to assist the physician 160 in the percutaneous access procedure (e.g., manipulating the needle 170 towards a target site). The display(s) 142 can also provide (e.g., via the augmented visualization interface 144 and/or another interface) information regarding the scope 120. For example, the control system 140 can receive real-time images that are captured by the scope 120 and display the real-time images via the display(s) 142. Additionally or alternatively, the control system 140 can receive signals (e.g., analog, digital, electrical, acoustic/sonic, pneumatic, tactile, hydraulic, etc.) from a medical monitor and/or a sensor associated with the patient 130, and the display(s) 142 can present information regarding the health or environment of the patient 130. Such information can include information that is displayed via a medical monitor including, for example, a heart rate (e.g., ECG, HRV, etc.), blood pressure/rate, muscle bio-signals (e.g., EMG), body temperature, blood oxygen saturation (e.g., SpO2), CO2, brainwaves (e.g., EEG), environmental and/or local or core body temperature, and so on.

To facilitate the functionality of the control system 140, the control system 140 can include various components (sometimes referred to as “subsystems”). For example, the control system 140 can include control electronics/circuitry, as well as one or more power sources, pneumatics, optical sources, actuators, memory/data storage devices, and/or communication interfaces. In some embodiments, the control system 140 includes control circuitry comprising a computer-based control system that is configured to store executable instructions, that when executed, cause various operations to be implemented. In some embodiments, the control system 140 is movable, such as that shown in FIG. 1, while in other embodiments, the control system 140 is a stationary system. Although various functionality and components are discussed as being implemented by the control system 140, any of this functionality and/or components can be integrated into and/or performed by other systems and/or devices, such as the robotic system 110, the table 150, and/or the EM generator 180 (or even the scope 120 and/or the needle 170). Example details of the control system 140 are discussed in further detail below in reference to FIG. 13.

The imaging device 190 can be configured to capture/generate one or more images of the patient 130 during a procedure, such as one or more x-ray or CT images. In examples, images from the imaging device 190 can be provided in real-time to view anatomy and/or medical instruments, such as the scope 120 and/or the needle 170, within the patient 130 to assist the physician 160 in performing a procedure. The imaging device 190 can be used to perform a fluoroscopy (e.g., with a contrast dye within the patient 130) or another type of imaging technique.

The various components of the medical system 100 can be communicatively coupled to each other over a network, which can include a wireless and/or wired network. Example networks include one or more personal area networks (PANs), local area networks (LANs), wide area networks (WANs), Internet area networks (IANs), cellular networks, the Internet, etc. Further, in some embodiments, the components of the medical system 100 are connected for data communication, fluid/gas exchange, power exchange, and so on, via one or more support cables, tubes, or the like.

Although various techniques and systems are discussed as being implemented as robotically-assisted procedures (e.g., procedures that at least partly use the medical system 100), the techniques and systems can be implemented in other procedures, such as in fully-robotic medical procedures, human-only procedures (e.g., free of robotic systems), and so on. For example, the medical system 100 can be used to perform a procedure without a physician holding/manipulating a medical instrument (e.g., a fully-robotic procedure). That is, medical instruments that are used during a procedure, such as the scope 120 and the needle 170, can each be held/controlled by components of the medical system 100, such as the robotic arm(s) 112 of the robotic system 110.

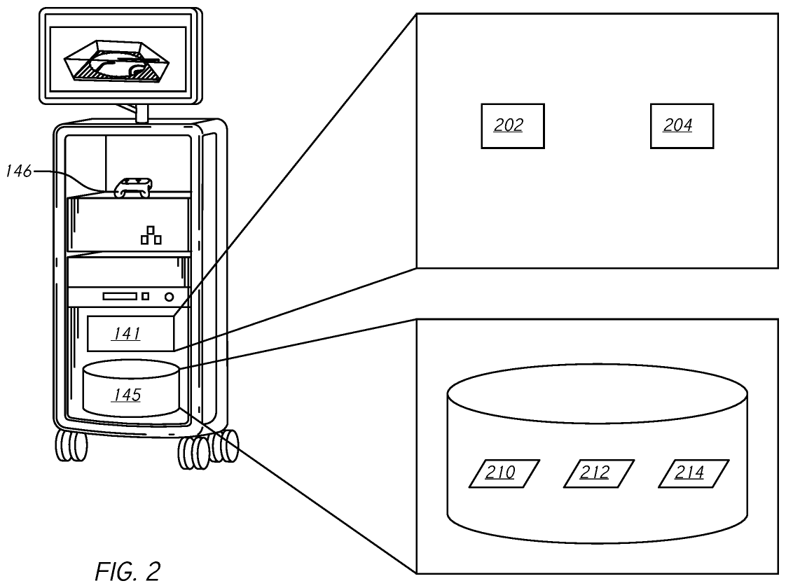

FIG. 2 is a block diagram illustrating the modules implemented by the augmentation module 141 and the data stored in the data store 145 of FIG. 1, according to an example embodiment. Referring first to the data shown in FIG. 2, the data store 145 shown in FIG. 2 includes two-dimensional data 210, location sensor data 212, robotic data 214, and instrument model data 216. The two-dimensional data may be image data acquired by the imaging device 190. For example, the two-dimensional data 210 may be data generate by or derived from a fluoroscope. The location sensor data 212 may be data generated or derived from sensors of any of the instruments used in the system 100. For example, the location sensor data 212 may include EM sensor data that specifies the 6-DoF location of the tip of the endoscope 120 or the needle 170. It is to be appreciated that the location sensor data 212 may specify a location relative to a coordinate frame of the sensor modality, as may be defined relative to an EM field generator or a reference point associated with a shape sensing fiber.

The robotic data 214 includes data regarding the kinematics of the instruments derived from commanded articulations, insertions/retractions. Examples of the robotic data 214 may include time series data specifying the commanded operation of the instruments, such as time series data specifying insertion commands, retraction commands, and articulation commands.

The instrument model data 216 may include data that models mechanics of one or more instruments that may be used by the system. Such models may include data that characterize how the instrument looks and data that characterizes how the instrument moves. Examples of instrument model data 216 include shape data, textures, moveable components, and the like.

In terms of the modules, the augmentation module 141 includes a segmentation module 202 and a data fusion module 204. The segmentation module 202 may operate on the two-dimensional image data 210 to generate segmented image data that segments the instruments depicted in the two-dimensional image data 210. The data fusion module 204 augments the two-dimensional image data 210 to include aspects of three-dimensional pose data regarding the instruments using system data. The operations of the modules are discussed in greater detail below.

### Three-Dimensional Tool Pose Estimation Methods and Operations

Details of the operations of exemplary instrument pose estimation systems are now discussed. The methods and operation disclosed herein are described relative to the instrument pose estimation systems 100 shown in FIG. 1 and the modules and other components shown in FIG. 2. However, it is to be appreciated that the methods and operations may be performed by any of the components, alone or in combination, discussed herein.

An instrument pose estimation system may generate a representation of a three-dimensional pose of instrument using data derived from a two-dimensional image (e.g., a fluoroscopy image) and location sensor data generated by a medical robotic system. In general, an instrument pose estimation system may (1) segment instruments from a two-dimensional image and (2) fuse three-dimensional location sensor data and robot data with the segmented two-dimensional image. Based on this fusion, an instrument pose estimation system may generate representations of the two-dimensional image with three dimensional representations of the instruments captured by the two-dimensional image. Examples of these representations, as contemplated in this disclosure, are discussed in greater detail below.

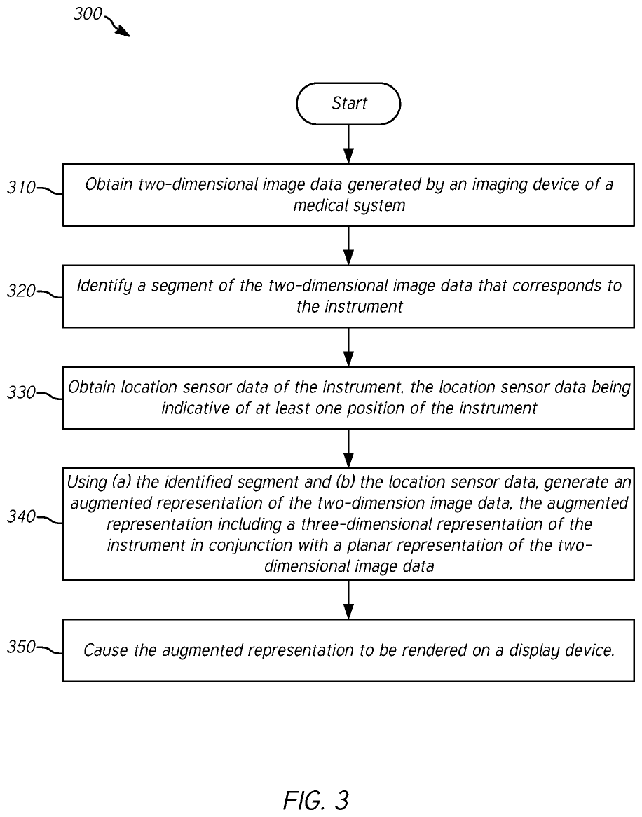

FIG. 3 is a flow-chart illustrating a method 300 to visualize a three-dimensional pose of a first instrument with respect to a two-dimensional image, according to an example embodiment. As FIG. 3 shows, the method 300 may begin at block 310, where the system 100 obtains two-dimensional image data generated by an imaging device. For example, during a medical procedure, a fluoroscope may capture an image that includes a representation of a patient's anatomy and the instruments in the captured area at the time the image was captured by the fluoroscope.

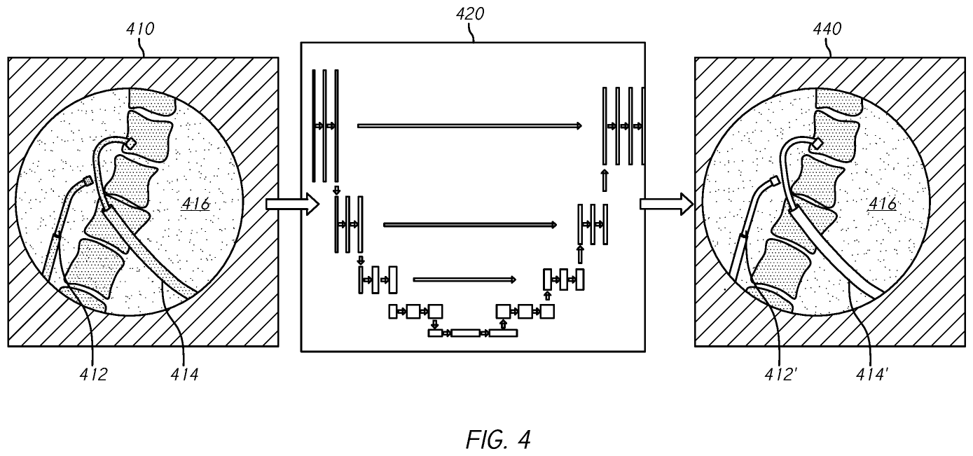

At block 320, the system 100 may identify a first segment of the two-dimensional image data that corresponds to the first instrument. In some embodiments, the system uses a neural network to perform the segmentation of block 320. It is to be appreciated that some embodiments of the system 100 may identify additional segments that correspond to different instruments. Thus, some embodiments may be capable of identifying multiple segments in two-dimensional image data that each correspond to different instruments. Identifying multiple instruments can be useful in embodiments that provide three-dimensional guidance for instruments that are attempting to rendezvous with each other. In some embodiments, identifying the first segment as corresponding to the first instrument may include obtaining the shape of the instruments in the two-dimensional image. Obtaining the shape may be useful where the sensor data of the instrument is reliable with respect to the shape of the instrument, as may be the case where a shape sensing fiber is used as a location sensor. Example embodiments of identifying the segments in the two-dimensional image is discussed in greater detail below with reference to FIG. 4.

At block 330, the system may obtain first location sensor data of the first instrument. The first location sensor data may be indicative of a position (or positions) of the first instrument. For example, in the case of EM sensors, the first location sensor data may be 6-DOF data indicative of a pose of the EM sensor within a coordinate frame defined by the field generator. As another example, in the case of shape sensing fibers, the first location sensor data may be strain data that can be processed to derive a shape of the shape sensing fiber. A location for the shape sensing fiber is then determined based on a coordinate frame of a known location of the shape sensing fiber. The first location sensor data may be obtained from the sensor data store of FIG. 2, and the first location sensor data may correspond to a time period that corresponds to a time in which the two-dimensional image data was generated.

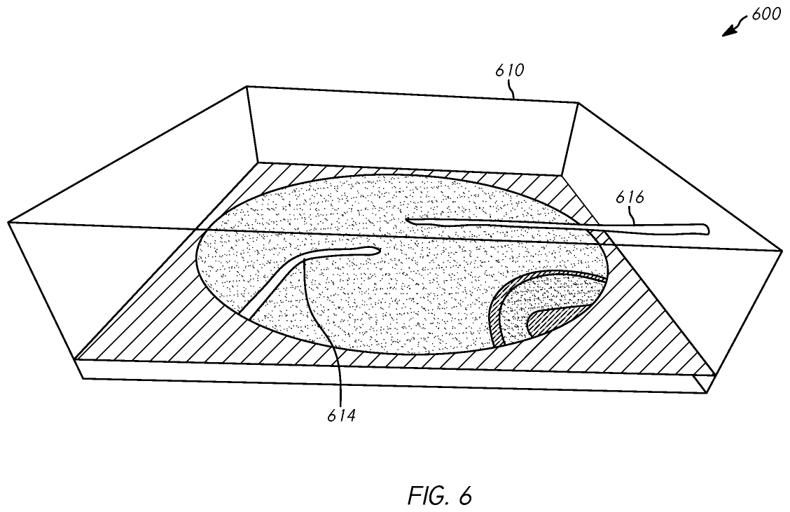

At block 340, the system 100 generates an augmented representation of the two-dimension image data using (a) the identified first segment and (b) the first location sensor data. In general, block 340 fuses the segmented two-dimensional data (e.g., block 320) with the location sensor data (e.g., block 330) to generate the augmented representation of the two-dimensional image data. In some embodiments, the augmented representation includes a three-dimensional representation of the first instrument in conjunction with a planar representation of the two-dimensional image data. An example embodiment of an augmented representation of the two-dimensional image data is described in greater detail below, with reference to FIG. 6.

At block 350, the system 100 causes the augmented representation to be rendered on a display device. The augmented representation of the two-dimensional image may be useful in a number of contexts. By way of example and not limitation, where an operator of the system is attempting to rendezvous two or more instruments but one of the instruments may lack vision capability. Another example is where the operator is in a blind driving situation where the operator is controlling an instrument that lacks vision capability itself or is outside the visible range of a camera. In these examples, and others, the augmented representation may provide the operator with three-dimensional context on the placement of the instruments that is not normally shown in the two-dimensional images.

### Segmentation of Two-Dimensional Image Data

As discussed above, with reference to block 320 of FIG. 3, the system 100 may identify one or more segments of the two-dimensional image data that correspond to the instruments depicted in the image. Details of embodiments for segmenting the images are now discussed in greater detail. FIG. 4 is a block diagram illustrating a segmentation of two-dimensional image data, according to an example embodiment. As FIG. 4 shows, a segmentation module 420 may receive two-dimensional image data 410 and may output two-dimensional segmented image data 440. The two-dimensional image data 410 may include image data generated or derived from a fluoroscope. The two-dimensional image data 410 includes data that, when rendered, depict an endoscope 412, a catheter 414, and patient anatomy 416. By way of example and not limitation, the catheter 414 may be entering a kidney through an endoluminal entrance. The catheter 414 may be entering the patient through a percutaneous entrance. Other embodiments of the two-dimensional image data may include data that varies the type, location, or number of the instruments or anatomy.

It is to be appreciated that although the rendered two-dimensional image data may visually depict the endoscope 412 and the catheter 414, the two-dimensional image data 410 itself may lack any sort of data that explicitly identifies where instruments may be located within the two-dimensional image data 410.

In contrast, the two-dimensional segmented image data 440 may include data that directly identifies the locations of the instruments within the two-dimensional image data 410. This data that directly identifies the locations of the instruments may be referred to as instrument segmentation data. The two-dimensional segmented image data 440 includes segmented data 412′ identifying the endoscope 412 and segmented data 414′ identifying the catheter 414. Although FIG. 4 depicts the two-dimensional segmented image data 440 as including both a visual component that depicts the endoscope 412, the catheter 414, and the anatomy 416, it is to be appreciated that the two-dimensional segmented image data 440 may lack a visual data component and instead only include meta data referencing the locations of the instruments within the two-dimensional image data 410. An example of such meta data may include a mask that maps pixel locations to classes identified in the segmentation.

As previously mentioned, the segmentation module 420 generates the two-dimensional segmented image data 440 from the two-dimensional image data 410. The segmentation module 420 may segment the two-dimensional data based on a multi-label pixel-wise segmentation. In an embodiment of a multi-label pixel-wise segmentation, pixels of a two-dimensional image can be assigned to belong to one or more classes. For example, where the system is segmenting based on two types of instruments, the segmentation module 420 may operate according to three classes, such as a first instrument class (e.g., an endoscope scope class), a second instrument class (e.g., a needle class), and a background or anatomy class. Other examples may operate with more or less classes of instruments that may depend on the types and number of instruments expected in a procedure.

The segmentation module 420 may apply a multi-class Unet (or similar convolutional neural network-based algorithm) for the segmentation of fluoro images. Assuming that the two-dimensional image 410 includes n×m pixels, the two-dimensional segmented image data 440 may be of size n×m×k, where k is the total number of instruments of interest plus background. In such an embodiment, if the two-dimensional segmented image data 440 includes n×m×k channels and the i-th channel corresponds to the needle segmentation, this channel will be associated with a threshold to obtain a binary mask of the needle. Although not shown, the two-dimensional segmented image data 440 may be postprocessed using morphological operations, connected component decomposition, smoothing, sharpening filters and other operations applicable to improve the quality of segmentation binary masks. The post-processing can be applied to all instrument channels to get individual segmentation masks.

The Unet-based segmentation can generate binary masks corresponding to each instrument present in the image. The next step may be for the system to recognize the planar geometry of each instrument. A neural network can be used to detect the working tips of each instrument using both input fluoro image and binary segmentation mask. The system can compute a gradient accumulation array using the edges of the binary mask and fluoro images. The idea of the gradient accumulation array is for each image pixel to estimate the number and strength of the image gradients that pass through this point. For cylindric and elliptic objects, many gradients originated at the object borders may intersect at the object center-centerline. A shape model of the needle will be fit into the needle segmentation mask using the detected tip and the estimated centerline as the anchor points. A deformable cylinder will be fit to the segmentation of the scope using the scope tip and centerline as the anchor points.

It is to be appreciated that the segmentation module can segment the instrument as a whole or the segmentation module can separately label articulation sections and the tip of the instruments (e.g., ureteroscope/percutaneous catheter) depending on the use case scenario. Segmenting the tip of the tool, for example, can be used to approximate a resolution of the image with respect to the visible anatomy by comparing the diameter and tip size of the scope in the segments and the reference diameter and size from the device specifications. Such device specifications can be acquired by the system when the tool is docked to a robotic arm. For example, a radio frequency identifier (RFID) tag on the tool may communicate a tool identifier that the system uses to lookup table to match tool identifiers to device specifications. As another example, the device specifications may be stored and communicated directly from the RFID tag of the tool. Using the device specifications and the segmentation of components of the tools, the system can determine scale information even if there's only one fluoro shot (0 degree anterior posterior). Using the diameter of the scope allows the system to derive information such as the depth or distance the tools/instruments are with respect to imaging device imaging plane.

In some embodiments, an initial two-dimensional image may not properly capture areas of interest with respect to the instruments used by the system. For example, some fluoroscopic images may fail to capture the working tips of the instruments. Embodiments discussed herein, may determine that such areas are missing from the segmentation and based on a known specification and or system data provide a recommendation to adjust the imaging device, such as rotating the C-arm some amount (e.g., 15, 30, 45 degrees) until tip is visible for accurate three-dimensional instrument reconstruction.

Fusing Two-Dimensional Segments with Three-Dimensional System Data

As discussed above, with reference to block 340 of FIG. 3, the system may fuse the segmented two-dimensional data with three-dimensional system data to generate an augmented representation of the two-dimensional image. In general, fusing the segmented two-dimension data with the three-dimensional system data may introduce a three-dimensional aspect to the instruments depicted in the two-dimensional image data. For simplicity of description, this section will discuss the fusing where the instrument segmentation identifies a needle instrument, a ureteroscope instrument, and a percutaneous catheter instrument. However, it is to be appreciated that this disclosure is not so limiting, and other embodiments may segment a different number of instruments and different kinds of instruments.

As discussed above, the segmentation step (e.g., block 320 of FIG. 3) obtains the shape of the instruments in the two-dimensional image. Some embodiments may then create centerlines of the segmented instrument models to be converted to 3D point clouds I_{N}, I_{U} and I_{P} with one unknown dimension. I_{N} represents the point cloud for the model of the needle instrument. I_{U} represents the point cloud for the model of the ureteroscope instrument. I_{P} represents the point cloud for the model of the percutaneous instrument.

The location sensor data from the needle and ureteroscope will be converted into 3D point clouds N and U, respectively, that represent the recent history of movements of the instruments. The history duration can depend on the visibility of the needle and ureteroscope. The approximate location p of the percutaneous instrument may be derived by reconstructing the commanded articulations, insertions/retractions. The united point cloud N∪U∪p will be transformed in order to fit the united point cloud I=I_{N} ∪I_{U} ∪I_{P} with one missing dimension.

To fit point clouds, some embodiments may determine a registration between the system data (e.g., the location sensor data, robot data, and the like) and the imaging device using a common coordinate frame. One such coordinate frame may include a patient or anatomy coordinate frame (simply referred to as a patient coordinate frame). To register the system data with the patient coordinate frame, some embodiments may rely on a determinable registration based on known mechanical linkages and kinematics of the robotic arms relative to the patient (or a platform supporting the patient, such as a bed). For example, a bed-based system may include robotic arms coupled to the base of the bed supporting the patient. In these bed-based embodiments, the transform between the robotic arms and the bed, and, in turn, the patient coordinate frame, is known kinematically from the robot data.

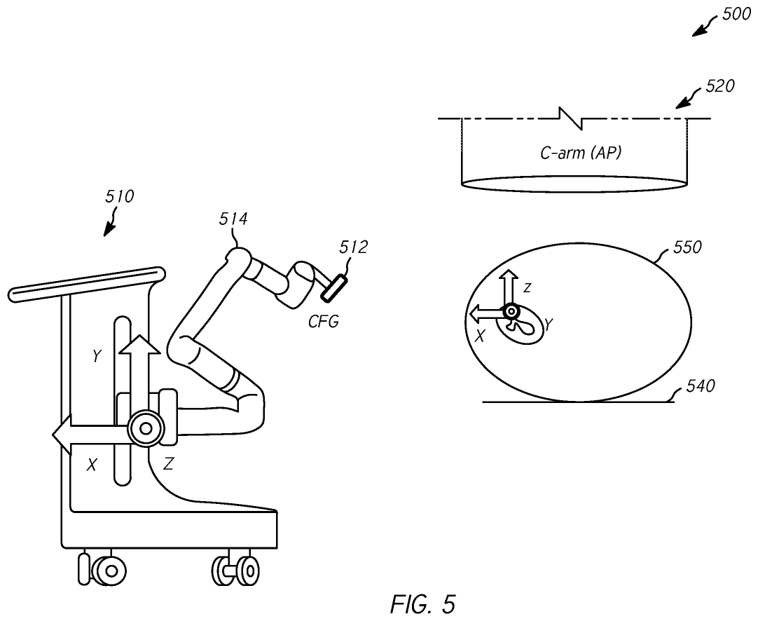

However, some embodiments may decouple the robotic elements from the bed supporting the patient. For example, in cart-based systems may allow the operator to position the robotic cart separately from the bed. In these cart-based embodiments, the system may rely on some assumptions regarding the position of the cart relative to the bed to determine a registration between the system data and the patient. For example, FIG. 5 is a diagram illustrating a cart system 500, according to an example embodiment. As FIG. 5 shows, the cart system 500 includes a robotic cart 510, an imaging device 520, a bed platform 540, and a patient 550. The robotic cart 510 may include an EM field generator 512 mounted to one of the robotic arms 514. Although not shown in FIG. 5, the robotic cart 510 may include additional arms for mounting and controlling instruments used to perform a medical procedure.

The position of the robotic cart 510 can be predetermined with respect to the bed platform 540. As the position is predetermined, the X-Y plane of the robot cart is parallel to the bed platform 540. The coordinates and orientation of the instruments mounted to the arms of the robotic cart 510 (e.g., needle and ureteroscope) are known in real time with respect to the EM field generator 512. EM field generator 512 pose is known with respect to the robot through kinematic data. Therefore, the positions of the instruments are known with respect to the robot coordinate frame. The commanded articulations are known from the instruments via kinematic data. The insertion/retraction commands are also known for the instruments. For instruments without location sensors, the position of those instruments can be approximated using the kinematic data (e.g., insertion, retraction, and articulation commands).

In terms of determining the transform from the two-dimensional imaging device to the patient coordinate frame, the acceptable transformations may depend on how the angle between the two-dimensional imaging device and the patient coordinate frame is determined. By way of example and not limitation, possible scenarios for determining the angle between the two-dimensional imaging device and the patient coordinate frame may include: a predetermined angle, an arbitrary known angle, or an arbitrary unknown angle. These scenarios are now discussed.

Predetermined angle: Some embodiments may define an angle in which the two-dimensional image is to be acquired from. One such angle may be the anteroposterior position (0 degree). In this case, the unknown dimension may correspond to dimension Z (up-down) in the robot's coordinate system. Having the image acquisition angle know, the registration process may restrict the transformations to rigid translations and scaling.

Arbitrary Known Angle: In some embodiments, the system may rely on an established access to the data from the imaging device and allow the user to acquire the two-dimensional image from a range of angles. As such, the operator of the system is not restricted on the angle from which the fluoro image needs to be acquired may have flexibility in selecting an angle for the given situation. As such, the system may include an interface for the allowing the module to receive the angle in which the two-dimensional angle has been acquired by the imaging device. For ease of discussion, the angle may be referred to as a. The point cloud I discussed above may then be rotated using angle α, so that the unknown dimension of the rotated J=T(I, α) cloud will also correspond to dimension Z in the robot's coordinate system. The transformations will also therefore be restricted to rigid translations and scaling.

Arbitrary Unknown Angle: In some embodiments, the two-dimensional image may be acquired from an angle α, but the angle α is not known to the system. For example, the system may lack an interface for the system to obtain the angle α. In such embodiments, the angle α will be included into the list of transformation in addition to rigid translation and scaling that is to be solved. There are a number of ways how the resulting alignment problem can be optimized. One of the approaches is to iteratively try different angles α. For a selected angle α, the point cloud is rotated J=T(I, α) so that its unknown dimension start to correspond to dimension Z in the robot's coordinate system. The transformations will be therefore restricted to rigid transformation and scaling. The obtain alignment quality measured using a similarity measure will be memorized for angle α. The next angle α will be selected for the analysis. Angles α can be tested hierarchically by starting from course angle search grid moving to a fine angle search grid. Alternatively, algorithms for point cloud registration that simultaneously optimize three dimensional rotations, translations and scaling can be used.

In each of the scenarios just discussed, the registration of points cloud N∪U ∪p and I=I_{N} ∪I_{U} ∪I_{P} may be restricted by the condition that the tip of the ureteroscope model I_{U} extracted from two-dimensional image shall match the last location detected by the location sensor of the ureteroscope U.

### Example Augmented Representations of Two-Dimensional Image Data

As described, with reference to block 340 of FIG. 3, the system may generate an augmented representation of two-dimensional image data. Embodiments of the augmented representation are now discussed in greater detail.

FIG. 6 is a diagram illustrating an example of an augmented representation 600 of two-dimensional image data, consistent with example embodiments contemplated by this disclosure. The augmented representation 600 may be a rendering of data that allows an operator of the system to visualize the three-dimensional locations and orientation of instruments captured in two-dimensional images, such as through fluoroscopy.

As shown, the augmented representation 600 includes a three-dimensional volume 610 that includes a representation of the two-dimensional image data 612 and representations of instruments 614, 616. The three-dimensional volume 610 may be a three-dimensional space within an understandable coordinate frame, such as a patient coordinate frame, a location sensor coordinate frame, an imaging device coordinate frame, or any other suitable coordinate frame. In some embodiments, the system may provide user interface elements to receive input from a user and change the orientation or perspective of the three-dimensional volume.

The representation of the two-dimensional image data 612 may be rendered as a planar image representing the two-dimensional image captured by the imaging device. For example, a fluoroscope image captured by an image device may be rendered

The representations of instruments 614, 616 may be renderings of instruments segmented from the two-dimensional image data but posed within the three-dimensional volume 610 according to the system data of the robotic system, such as the location sensor data and the robot data. In some embodiments, the system may render the representations of the instruments 614, 616 according to instrument renderings accessible to the system. For example, some embodiments may maintain a database of computerized models of the instruments. In such embodiments, the system may be configured to modify the figures according to the system data (e.g., the location sensor data and/or robot data).

It is to be appreciated that the augmented representation 600 may provide spatial awareness the instruments (e.g., instruments 614, 616) with respect to the anatomy or each other based on a two-dimensional image and system data captured by the system. This may be a particular advantage in that the methods performed here may achieve such spatial awareness with comparatively fewer steps in the workflow by avoiding many steps normally provided to register the coordinate frames of two different modalities. Further, such spatial awareness may be provided in context of a medium that the operator of the system is accustomed to using, such as fluoroscopy.

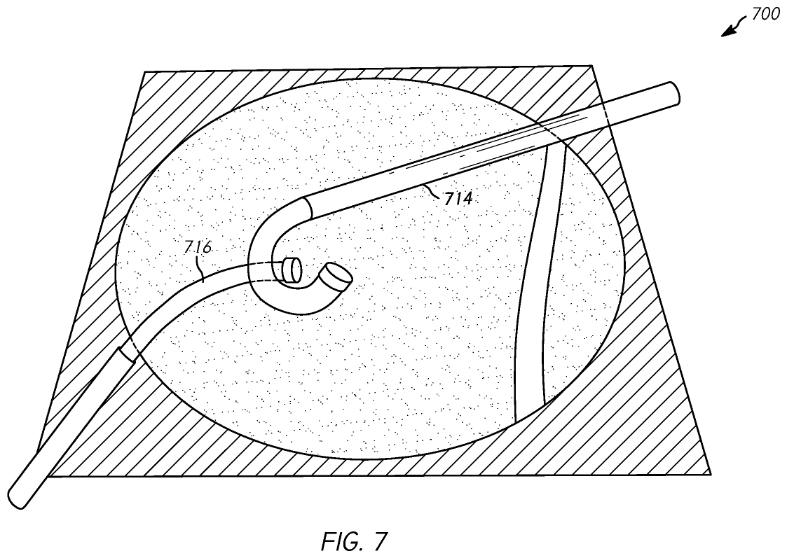

Other representations are possible here. For example, FIG. 7 is a diagram illustrating an example of an augmented representation 700 of two-dimensional image data, consistent with example embodiments contemplated by this disclosure. The augmented representation 700 may be a rendering of data that allows an operator of the system to visualize the three-dimensional locations and orientation of instruments captured in two-dimensional images, such as through fluoroscopy. Compared to the augmented representation 600 of FIG. 6, the augmented representation 700 may lack a three-dimensional volume.

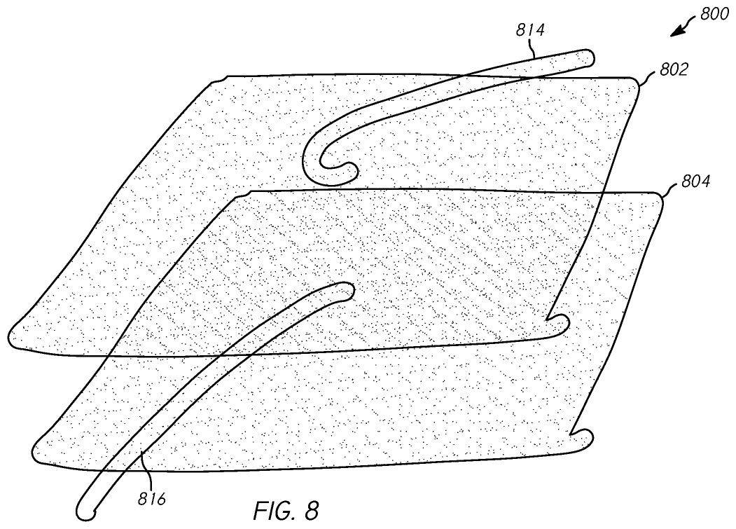

FIG. 8 is a diagram illustrating an example of an augmented representation 800 of two-dimensional image data, consistent with example embodiments contemplated by this disclosure. The augmented representation 800 may be a rendering of data that allows an operator of the system to visualize the three-dimensional locations and orientation of instruments captured in two-dimensional images, such as through fluoroscopy. The augmented representation 800 may include different planes 802, 804 that are rooted to some element of the instruments 814, 816 respectively.

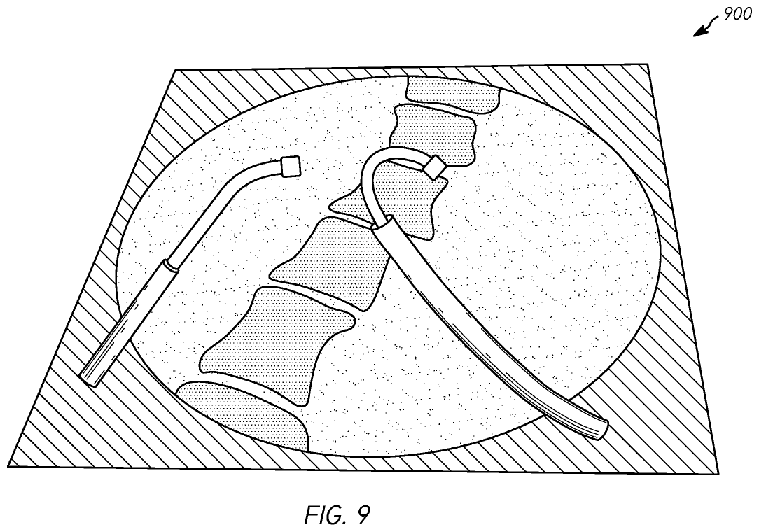

FIG. 9 is a diagram illustrating an example of an augmented representation 900 of two-dimensional image data, consistent with example embodiments contemplated by this disclosure.

### Segmented Shape as Input to Other Subsystems

In some embodiments, the output of the segmentation of the two-dimensional images may be used as an input to other sub-systems. For example, the robotic control of an instrument may receive the segmentation with may include a shape of the instrument and, in some cases, pose relative to anatomy. The robotic control can then use the shape and/or pose in the form of a closed-feedback loop for the instrument to achieve a desired pose or to navigate to a given location. As further examples, a navigation system may use the shape of the instrument and/or relative pose as input into one or more localization algorithms. To illustrate, in cases where the segmented shape differs from the kinematic model, the navigation system may lower the confidence level of the robotic localization algorithm. Some systems may also use the shape to detect system status, such as buckling events and the like.

Finally, the segmented shape of the instruments can be used to better locate the instrument within an anatomy. For example, with a known internal shape of an anatomy (as may be determined based on a three-dimensional model or two-dimensional segmentation), the systems described herein may fit the known anatomy to the segmented shape.

### Implementing Systems and Terminology

Implementations disclosed herein provide systems, methods and apparatus for augmenting a two-dimensional image with three-dimensional pose information of instruments shown in the two-dimensional image.

The systems described herein can include a variety of other components. For example, the systems can include one or more control electronics/circuitry, power sources, pneumatics, optical sources, actuators (e.g., motors to move the robotic arms), memory, and/or communication interfaces (e.g. to communicate with another device). In some embodiments, the memory can store computer-executable instructions that, when executed by the control circuitry, cause the control circuitry to perform any of the operations discussed herein. For example, the memory can store computer-executable instructions that, when executed by the control circuitry, cause the control circuitry to receive input and/or a control signal regarding manipulation of the robotic arms and, in response, control the robotic arms to be positioned in a particular arrangement.

The various components of the systems discussed herein can be electrically and/or communicatively coupled using certain connectivity circuitry/devices/features, which can or may not be part of the control circuitry. For example, the connectivity feature(s) can include one or more printed circuit boards configured to facilitate mounting and/or interconnectivity of at least some of the various components/circuitry. In some embodiments, two or more of the control circuitry, the data storage/memory, the communication interface, the power supply unit(s), and/or the input/output (I/O) component(s), can be electrically and/or communicatively coupled to each other.

The term “control circuitry” is used herein according to its broad and ordinary meaning, and can refer to any collection of one or more processors, processing circuitry, processing modules/units, chips, dies (e.g., semiconductor dies including come or more active and/or passive devices and/or connectivity circuitry), microprocessors, micro-controllers, digital signal processors, microcomputers, central processing units, graphics processing units, field programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions. Control circuitry can further comprise one or more, storage devices, which can be embodied in a single memory device, a plurality of memory devices, and/or embedded circuitry of a device. Such data storage can comprise read-only memory, random access memory, volatile memory, non-volatile memory, static memory, dynamic memory, flash memory, cache memory, data storage registers, and/or any device that stores digital information. It should be noted that in embodiments in which control circuitry comprises a hardware state machine (and/or implements a software state machine), analog circuitry, digital circuitry, and/or logic circuitry, data storage device(s)/register(s) storing any associated operational instructions can be embedded within, or external to, the circuitry comprising the state machine, analog circuitry, digital circuitry, and/or logic circuitry.

The term “memory” is used herein according to its broad and ordinary meaning and can refer to any suitable or desirable type of computer-readable media. For example, computer-readable media can include one or more volatile data storage devices, non-volatile data storage devices, removable data storage devices, and/or nonremovable data storage devices implemented using any technology, layout, and/or data structure(s)/protocol, including any suitable or desirable computer-readable instructions, data structures, program modules, or other types of data.

Computer-readable media that can be implemented in accordance with embodiments of the present disclosure includes, but is not limited to, phase change memory, static random-access memory (SRAM), dynamic random-access memory (DRAM), other types of random access memory (RAM), read-only memory (ROM), electrically erasable programmable read-only memory (EEPROM), flash memory or other memory technology, compact disk read-only memory (CD-ROM), digital versatile disks (DVD) or other optical storage, magnetic cassettes, magnetic tape, magnetic disk storage or other magnetic storage devices, or any other non-transitory medium that can be used to store information for access by a computing device. As used in certain contexts herein, computer-readable media may not generally include communication media, such as modulated data signals and carrier waves. As such, computer-readable media should generally be understood to refer to non-transitory media.

### Additional Embodiments

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is understood with the context as used in general to convey that an item, term, element, etc. may be either X, Y, or Z. Thus, such conjunctive language is not generally intended to imply that certain embodiments require at least one of X, at least one of Y, and at least one of Z to each be present.

It should be appreciated that in the above description of embodiments, various features are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the disclosure should not be limited by the particular embodiments described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “using” or “based on” a condition, event, or data may also be performed based on one or more other conditions, events, or data not explicitly recited.

Unless otherwise defined, all terms (including technical and scientific terms) used herein have the same meaning as commonly understood by one of ordinary skill in the art to which example embodiments belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

1. A method to visualize a three-dimensional pose of an instrument with respect to a two-dimensional image, the method comprising:
obtaining two-dimensional image data generated by an imaging device of a medical system;
identifying a segment of the two-dimensional image data that corresponds to the instrument;
obtaining location sensor data of the instrument, the location sensor data being indicative of at least one position of the instrument;
using (a) the identified segment and (b) the location sensor data, generating an augmented representation of the two-dimensional image data, the augmented representation including a three-dimensional representation of the instrument in conjunction with a planar representation of the two-dimensional image data; and
causing the augmented representation to be rendered on a display device.

2. The method of claim 1, further comprising:
identifying another portion of the two-dimensional image data that corresponds to an another instrument; and
obtaining additional location sensor data indicative of at least one position of the another instrument, wherein:
generating the augmented representation of the two-dimension image data is further based on (c) the identified another portion and (d) the additional location sensor data, and
the augmented representation further includes a three-dimensional representation of the another instrument in conjunction with the planar representation of the two-dimensional image data.

3. The method of claim 1, wherein the augmented representation further includes:
a first indicator representing a plane anchored at a position of the instrument; and
a second indicator representing a plane anchored at a position of the another instrument.

4. (canceled)

5. The method of claim 1, wherein the location sensor data includes sensor data generated by a location sensor located at least partially on a distal end of the instrument.

6. (canceled)

7. (canceled)

8. The method of claim 1, wherein the location sensor data includes data from a kinematic model of the instrument, the kinematic model being derived based on commanded movement of the instrument.

9. The method of claim 1, wherein the two-dimensional image data lacks a third dimension, and generating the augmented representation further comprises:
determining an angle of the two-dimension image data relative to a coordinate frame of the location sensor data; and
based on the angle and the location sensor data, adding data indicative of the instrument to the two-dimensional image data, the added data includes positioning in the third dimension.

10. (canceled)

11. (canceled)

12. (canceled)

13. The method of claim 1, further comprising:
generating first point cloud data based on the location sensor data, the first point cloud data including first point data in a first dimension, a second dimension, and a third dimension; and
generating second point cloud data based on the two-dimensional image data, the second point cloud data including second point data in the first dimension, the second dimension, and an unknown dimension, wherein generating the three-dimensional representation of the two-dimension image data further comprises using the first point cloud data to update the second point cloud data to include third point cloud data in the unknown dimension.

14. (canceled)

15. (canceled)

16. (canceled)

17. A medical system to visualize a three-dimensional pose of an instrument with respect to a two-dimensional image, the medical system comprising:
an imaging device to generate two-dimensional image data;
a memory that stores computer-executable instructions that, when executed by a control circuitry, cause the control circuitry to perform:
identify a segment of the two-dimensional image data that corresponds to the instrument;
obtain location sensor data of the instrument, the location sensor data being indicative of at least one position of the instrument;
use (a) the identified segment and (b) the location sensor data to generate an augmented representation of the two-dimensional image data, the augmented representation including a three-dimensional representation of the instrument in conjunction with a planar representation of the two-dimensional image data; and
cause the augmented representation to be rendered on a display device.

18. The medical system of claim 17, wherein the computer-executable instructions further cause the control circuitry to perform:
identify another portion of the two-dimensional image data that corresponds to an another instrument; and
obtain additional location sensor data indicative of at least one position of the another instrument, wherein:
generate the augmented representation of the two-dimension image data is further based on (c) the identified another portion and (d) the additional location sensor data, and
the augmented representation further includes a three-dimensional representation of the another instrument in conjunction with the planar representation of the two-dimensional image data.

19. The medical system of claim 17, wherein the augmented representation further includes:
a first indicator representing a plane anchored at a position of the instrument; and
a second indicator representing a plane anchored at a position of the another instrument.

20. (canceled)

21. The medical system of claim 17, wherein the location sensor data includes sensor data generated by a location sensor located at least partially on a distal end of the instrument.

22. (canceled)

23. (canceled)

24. The medical system of claim 17, wherein the location sensor data includes data from a kinematic model of the instrument, the kinematic model being derived based on commanded movement of the instrument.

25. The medical system of claim 17, wherein the two-dimensional image data lacks a third dimension, and generate the augmented representation further comprises:
determine an angle of the two-dimension image data relative to a coordinate frame of the location sensor data; and
based on the angle and the location sensor data, add data indicative of the instrument to the two-dimensional image data, the added data includes positioning in the third dimension.

26. (canceled)

27. (canceled)

28. (canceled)

29. The medical system of claim 17, generate first point cloud data based on the location sensor data, the first point cloud data including first point data in a first dimension, a second dimension, and a third dimension; and
generate second point cloud data based on the two-dimensional image data, the second point cloud data including second point data in the first dimension, the second dimension, and an unknown dimension, wherein generate the three-dimensional representation of the two-dimension image data further comprises use the first point cloud data to update the second point cloud data to include third point cloud data in the unknown dimension.

30. (canceled)

31. (canceled)

32. (canceled)

33. A non-transitory computer readable storage medium having stored thereon instructions that, when executed, cause a processor of a device to at least:
obtain two-dimensional image data generated by an imaging device of a medical system;
identify a segment of the two-dimensional image data that corresponds to an instrument;
obtain location sensor data of the instrument, the location sensor data being indicative of at least one position of the instrument;
use (a) the identified segment and (b) the location sensor data to generate an augmented representation of the two-dimensional image data, the augmented representation including a three-dimensional representation of the instrument in conjunction with a planar representation of the two-dimensional image data; and
cause the augmented representation to be rendered on a display device.

34. The non-transitory computer readable storage medium of claim 33, wherein the instructions further cause the processor to:
identify another portion of the two-dimensional image data that corresponds to an another instrument; and
obtain additional location sensor data indicative of at least one position of the another instrument, wherein:
generate the augmented representation of the two-dimension image data is further based on (c) the identified another portion and (d) the additional location sensor data, and
the augmented representation further includes a three-dimensional representation of the another instrument in conjunction with the planar representation of the two-dimensional image data.

35. The non-transitory computer readable storage medium of claim 33, wherein the augmented representation further includes:
a first indicator representing a plane anchored at a position of the instrument; and
a second indicator representing a plane anchored at a position of the another instrument.

36. (canceled)

37. The non-transitory computer readable storage medium of claim 33, wherein the location sensor data includes sensor data generated by a location sensor located at least partially on a distal end of the instrument.

38. (canceled)

39. (canceled)

40. (canceled)

41. The non-transitory computer readable storage medium of claim 33, wherein the two-dimensional image data lacks a third dimension, and generate the augmented representation further comprises:
determine an angle of the two-dimension image data relative to a coordinate frame of the location sensor data; and
based on the angle and the location sensor data, add data indicative of the instrument to the two-dimensional image data, the added data includes positioning in the third dimension.

42. (canceled)

43. (canceled)

44. (canceled)

45. The non-transitory computer readable storage medium of claim 33, generate first point cloud data based on the location sensor data, the first point cloud data including first point data in a first dimension, a second dimension, and a third dimension; and
generate second point cloud data based on the two-dimensional image data, the second point cloud data including second point data in the first dimension, the second dimension, and an unknown dimension, wherein generate the three-dimensional representation of the two-dimension image data further comprises use the first point cloud data to update the second point cloud data to include third point cloud data in the unknown dimension.

46. (canceled)

47. (canceled)

48. (canceled)
